(19) United States
(12) Patent Application Publication
(10) Pub. No.: US 2013/0066359 A1
(43) Pub. Date: Mar. 14, 2013

(54) VASO-OCCLUSIVE DEVICE

(71) Applicants: Richard Murphy, Sunnyvale, CA (US); Hancun Chen, San Ramon, CA (US)

(72) Inventors: Richard Murphy, Sunnyvale, CA (US); Hancun Chen, San Ramon, CA (US)

(73) Assignees: STRYKER NV OPERATIONS LIMITED, Dublin (IE); STRYKER CORPORATION, Kalamazoo, MI (US)

(21) Appl. No.: 13/608,505

(22) Filed: Sep. 10, 2012

Related U.S. Application Data
(60) Provisional application No. 61/533,944, filed on Sep. 13, 2011.

(51) Int. Cl. A61F 2/01; B21D 31/00

(52) U.S. Cl. 606/200; 72/364

(57) ABSTRACT

A vaso-occlusive device includes a coil made from coil wire, wherein the coil wire includes a core containing a core material having a first electrical potential covered by a coating containing a coating material having a second electrical potential, wherein the second electrical potential is less than the first electrical potential. A method of manufacturing a vaso-occlusive device includes cladding a core with a magnesium containing coating to form a wire, wrapping the wire around a mandrel to form a coil, and heat-setting the coiled wire. A method of occluding a body cavity with a vaso-occlusive device includes delivering the vaso-occlusive device to a body cavity and inducing a reduction reaction in the body cavity and adjacent the vaso-occlusive device, wherein the reduction reaction reduces infections and promotes cell proliferation in the body cavity.

RELATED APPLICATION DATA

The present application claims the benefit under 35 U.S.C. § 119 to U.S. provisional patent application Ser. No. 61/533,944, filed Sep. 13, 2011. The foregoing application is hereby incorporated by reference into the present application in its entirety.

### FIELD

The field of the disclosed inventions generally relates to vaso-occlusive devices for establishing an embolus or vascular occlusion in a vessel of a human or veterinary patient. More particularly, the disclosed inventions relate to vaso-occlusive coils including metals having low electrical potential.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference, describes a vaso-occlusive device that assumes a linear, helical primary shape when stretched for placement through the lumen of a delivery catheter, and a folded, convoluted secondary shape when released from the delivery catheter and deposited in the vasculature.

In order to deliver the vaso-occlusive devices to a desired site in the vasculature, e.g., within an aneurysmal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire is extended out of the distal end opening of the micro-catheter and into the aneurysm. Once in the aneurysm, the vaso-occlusive devices bend to allow more efficient and complete packing The vaso-occlusive device is then released or “detached” from the end delivery wire, and the delivery wire is withdrawn back through the catheter. Depending on the particular needs of the patient, one or more additional occlusive devices may be pushed through the catheter and released at the same site.

One well-known way to release a vaso-occlusive device from the end of the pusher wire is through the use of an electrolytically severable junction, which is a small exposed section or detachment zone located along a distal end portion of the pusher wire. The detachment zone is typically made of stainless steel and is located just proximal of the vaso-occlusive device. An electrolytically severable junction is susceptible to electrolysis and disintegrates when the pusher wire is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

Existing vaso-occlusive devices can be coated with liquid or gel therapeutic agents to impart antimicrobial and/or cellular grow enhancing properties. However, such liquid or gel coatings complicate the manufacture, packaging, and use of vaso-occlusive devices. Further the coatings may be dislodged during delivery through narrow catheters. Accordingly, there is a need for vaso-occlusive devices having antimicrobial and cellular grow enhancing properties without the need for liquid or gel coatings.

### SUMMARY

In one embodiment of the disclosed inventions, a vaso-occlusive device includes a coil made from coil wire, wherein the coil wire includes a core containing a core material having a first electrical potential covered by a coating containing a coating material having a second electrical potential, wherein the second electrical potential is less than the first electrical potential. In some embodiments, the second electrical potential is at least 20% less than the first electrical potential.

In other embodiments, the coating material is magnesium. In some embodiments, the coating includes pure magnesium. In other embodiments, the coating includes a magnesium alloy, such as a magnesium-iron alloy. Additionally or alternatively, the coating includes porous magnesium.

In some embodiments, the coating is clad onto the core, which may include biologically compatible polymers, biologically compatible ceramics, or biologically compatible metals. The biologically compatible metal may be selected from the group consisting of platinum, platinum-tungsten alloy, platinum-iridium alloy, platinum-rhenium alloy, and platinum-palladium alloy.

In embodiments where the core material is a biologically compatible polymer or a biologically compatible ceramic, the core may also include a radiopaque agent, such as barium particles, barium fibers, iodine particles, iodine particles, tungsten particles, tungsten fibers, platinum particles, or platinum fibers. In other such embodiments, the coating may also include a radiopaque agent, such as barium, iodine, tungsten, or platinum. In still other embodiments, the coating includes nano pores have a lubricious liquid disposed therein.

In another embodiment of the disclosed inventions, a method of manufacturing a vaso-occlusive device including a coil made from coil wire including a core containing a core material having a first electrical potential covered by a coating containing a coating material having a second electrical potential, wherein the second electrical potential is less than the first electrical potential, includes cladding the core with the coating to form a wire, wrapping the wire around a mandrel to form a coil, and heat-setting the coiled wire. The coating material may be magnesium.

In yet another embodiment of the disclosed inventions, a method of occluding a body cavity with a vaso-occlusive device including a core containing a core material having a first electrical potential covered by a coating containing a coating material having a second electrical potential, wherein the second electrical potential is less than the first electrical potential, includes delivering the vaso-occlusive device to a body cavity and inducing a reduction reaction in the body cavity and adjacent the vaso-occlusive device, wherein the reduction reaction reduces infections and promotes cell proliferation in the body cavity. The method may also include, delivering a second vaso-occlusive device to the body cavity and inducing a second reduction reaction in the body cavity and adjacent the second vaso-occlusive device, wherein the second vaso-occlusive device does not contain the coating material, and wherein the second reduction reaction reduces infections and promotes cell proliferation in the body cavity.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale. The relative scale of select elements may have been exaggerated for clarity. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIGS. 1-3 are detailed longitudinal cross-section views of vaso-occlusive coils constructed according to various embodiments of the disclosed inventions.

FIG. 4 is a detailed cross-sectional view of a coil wire according to an embodiment of the disclosed inventions.

FIG. 5 is a perspective view of a vaso-occlusive coil in a natural state mode, illustrating one exemplary secondary configuration according to an embodiment of the disclosed inventions.

### DETAILED DESCRIPTION OF THE ILLUSTRATED EMBODIMENTS

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment of the disclosed inventions needs not have all the aspects or advantages shown. An aspect or an advantage described in conjunction with a particular embodiment of the disclosed inventions is not necessarily limited to that embodiment and can be practiced in any other embodiments even if not so illustrated.

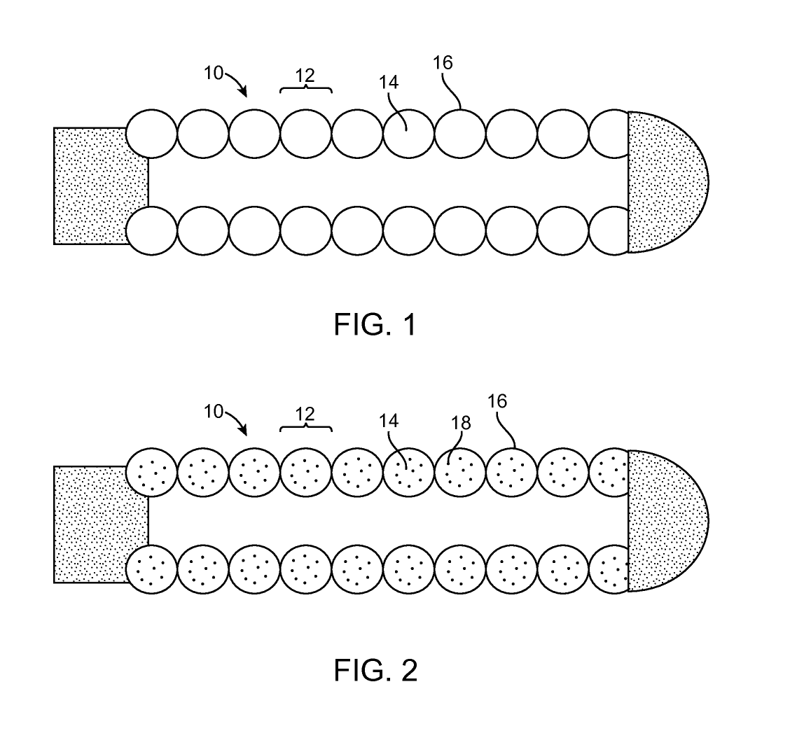

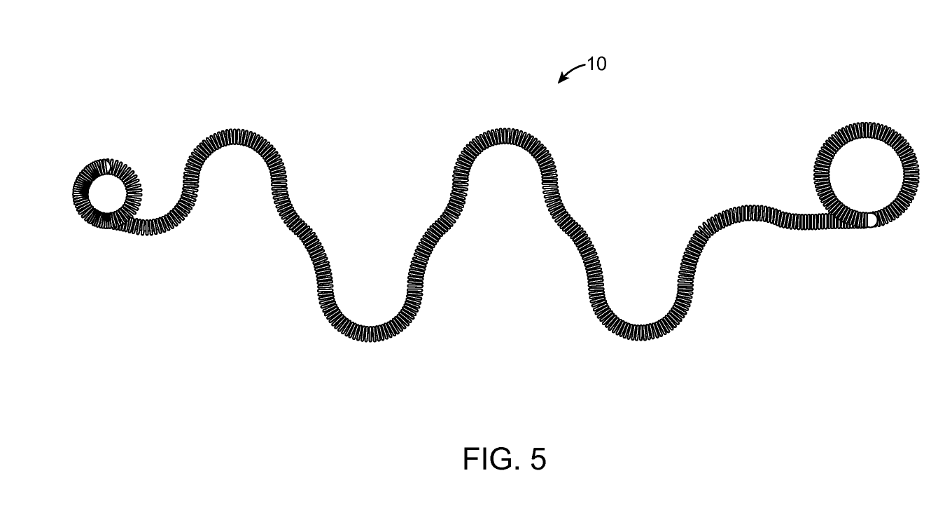

FIG. 1 illustrates a vaso-occlusive coil 10 in accordance with one embodiment. The vaso-occlusive coil 10 is formed by winding coil wire 12 over mandrel and heat-setting the wire 12 to set a secondary shape (see FIG. 5). The coil wire 12 has a core 14 surrounded by a coating 16. The core 14 is made of a platinum-tungsten alloy and the coating 16 is made of magnesium.

In general, the core 14 can be made from any suitable biologically compatible material. For example, the core 14 may be made from a biologically compatible polymer, a biologically compatible ceramic, and/or a biologically compatible metal. Suitable biologically compatible metals include platinum, platinum-tungsten alloy, platinum-iridium alloy, platinum-rhenium alloy, and platinum-palladium alloy. A suitable platinum-tungsten alloy is 8% tungsten and the remainder platinum.

While the coating 16 in this embodiment contains magnesium, other biocompatible metals having a lower electrical potential than the core material, such as zinc and stainless steel, can also be used as the coating material. The electrical potential of the coating material is preferably at least 20% lower than the electrical potential of the core material. More preferably, the electrical potential of the coating material is at least 100% lower than the electrical potential of the core material. Still more preferably, the electrical potential of the coating material is at least 150% lower than the electrical potential of the core material. Even more preferably, the electrical potential of the coating material is at least 900% lower than the electrical potential of the core material. For instance, the electrical potential of magnesium in the coating 16 is approximately 900% lower than the electrical potential of platinum in the core 14. Further, the electrical potential of stainless steel in the coating 16 is approximately 150% lower than the electrical potential of platinum in the core 14.

The magnesium in the coating 16 induces a strong reduction reaction near the vaso-occlusive coil 10, which draws down the electrical potential to a large negative value, which has antimicrobial properties (reducing infections) and cell proliferation enhancing properties in vivo.

Such properties are desirable in, for example, the treatment of aneurysms, because reducing infection and enhancing cellular grow into the aneurysm would promote healing of the aneurysm. Aneurysms may also be treated by packing with a mixture of uncoated coils (e.g., platinum coils) and coils with a Magnesium containing coating 16. When a platinum coil is near a magnesium coated coil, the magnesium can also draw down the electrical potential to a large negative value near the platinum coil, thereby imparting antimicrobial and cell proliferation properties to the platinum coil.

In embodiments where the core 14 is made from radiolucent materials such as biologically compatible polymers and ceramics, radiopaque agents 18 are incorporated into the vaso-occlusive coil 10. For instance, the core 14 of the vaso-occlusive coil 10 shown in FIG. 2 is made of a biologically compatible polymer with a radiopaque agent 18 added into the polymer matrix. Suitable radiopaque agents 18 include barium particles, barium fibers, iodine particles, iodine particles, tungsten particles, tungsten fibers, platinum particles, and platinum fibers.

In the embodiment in FIG. 3, the core 14 is made of a biologically compatible polymer or ceramic. In order to render the vaso-occlusive coil 10 radiopaque, the coating 16 is made from co-clad bi-metal including magnesium and a radiopaque metal, such as magnesium-barium, magnesium-iodine, magnesium-tungsten or magnesium-platinum.

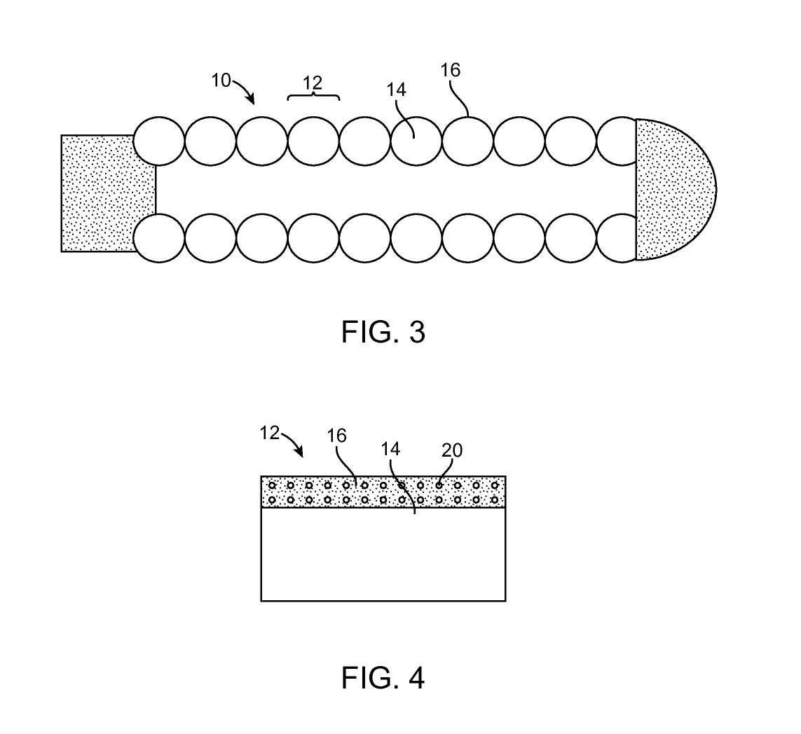

As shown in FIG. 4, the coating 16 may preferably have nano pores 20 in it. These nano pores 20 may contain a lubricious liquid to improve device deliverability.

In some embodiments, the vaso-occlusive coils 10 described herein may have the simple linear shape shown previously, or may have shapes which are more complex. FIG. 5 shows what is termed a “secondary” shape in that it is formed from the primary coil by winding the primary coil on a form of a desired shape, e.g. a mandrel, and then heat treating the so-formed shape. Various other secondary shapes may be implemented in embodiments of the vaso-occlusive coil 10 described herein.

In addition to the variations described above, the vaso-occlusive coils 10 of the disclosed inventions can be wound from coil wires 12 that differ in other characteristics, as long as the coil wires 12 include a magnesium coating. For instance, the coil wire 12 may have various cross-sectional geometries, such as circular, oval, triangular, and square. Further, the coil wire 12 may have flattened sections with a short cross-sectional axis. The vaso-occlusive coil 10 is more flexible, i.e., likely to bend, where the short cross-sectional axis is perpendicular to the longitudinal axis of the vaso-occlusive coil 10.

Moreover, the vaso-occlusive coil 10 may be co-wound from a plurality of coil wires 12, as along as at least one of the wires 12 includes a magnesium coating. While the above-described embodiments of FIGS. 1-5 are directed to single layer vaso-occlusive coils 10, it should be appreciated by those skilled in the art that double-coil embodiments, i.e., those having an outer coil layer and an inner coil layer may be included in alternative embodiments, in accordance with the inventive aspects disclosed herein. In double-coil embodiments, the outer coil layer includes a magnesium coating.

In any of the embodiments described herein, the coil wire 12 may have a cross-sectional dimension that is in the range of 0.00002 and 0.01 inches. The coil loops formed by the coil wire 12 may have a cross-sectional dimension between 0.003 and 0.03 inches. For neurovascular applications, the diameter of the coil loops may be anywhere from 0.008 to 0.025 inches, preferably from 0.009-0.015 inches. In other embodiments, the coil wire 12 may have other cross-sectional dimensions, and the coil loops may have other cross-sectional dimensions. In some embodiments, the coil wire 12 for forming the coil loops should have a sufficient diameter to provide a hoop strength to the resulting vaso-occlusive coil 10 sufficient to hold the coil 10 in place within the chosen body site, lumen or cavity, without substantially distending the wall of the site and without moving from the site as a result of the repetitive fluid pulsing found in the vascular system.

In any of the embodiments described herein, the axial length of the vaso-occlusive device 10 may be in the range of 0.5 to 100 cm, and more preferably, in the range of 1.0 to 60 cm. Depending upon use, the vaso-occlusive coil 10 may have 10-75 turns per centimeter, or more preferably 10-40 turns per centimeter. In other embodiments, the vaso-occlusive coil 10 may have other lengths and/or other number of turns per centimeter.

This completes the description of the preferred and alternate embodiments of the invention. Those skilled in the art may recognize other equivalents to the specific embodiment described herein which equivalents are intended to be encompassed by the claims attached hereto.

What is claimed is:

1. A vaso-occlusive device, comprising a coil made from coil wire, wherein the coil wire includes a core containing a core material having a first electrical potential covered by a coating containing a coating material having a second electrical potential, wherein the second electrical potential is less than the first electrical potential.

2. The vaso-occlusive device of claim 1, wherein the second electrical potential is at least 20% less than the first electrical potential.

3. The vaso-occlusive device of claim 1, wherein the coating material is magnesium.

4. The vaso-occlusive device of claim 3, wherein the coating comprises pure magnesium.

5. The vaso-occlusive device of claim 3, wherein the coating comprises a magnesium alloy.

6. The vaso-occlusive device of claim 3, wherein the coating comprises porous magnesium.

7. The vaso-occlusive device of claim 3, wherein the coating comprises a magnesium-iron alloy.

8. The vaso-occlusive device of claim 1, wherein the coating is clad onto the core.

9. The vaso-occlusive device of claim 1, wherein the core material is selected from the group consisting of biologically compatible polymers, biologically compatible ceramics, and biologically compatible metals.

10. The vaso-occlusive device of claim 9, wherein the core material is a biologically compatible metal selected from the group consisting of platinum, platinum-tungsten alloy, platinum-iridium alloy, platinum-rhenium alloy, and platinum-palladium alloy.

11. The vaso-occlusive device of claim 9, wherein the core material is a biologically compatible polymer or a biologically compatible ceramic, the core further comprising a radiopaque agent.

12. The vaso-occlusive device of claim 11, wherein the radiopaque agent is selected from the group consisting of barium particles, barium fibers, iodine particles, iodine particles, tungsten particles, tungsten fibers, platinum particles, and platinum fibers.

13. The vaso-occlusive device of claim 9, wherein the core material is a biologically compatible polymer or a biologically compatible ceramic, the coating further comprising a radiopaque agent.

14. The vaso-occlusive device of claim 13, wherein the radiopaque agent is selected from the group consisting of barium, iodine, tungsten, and platinum.

15. The vaso-occlusive device of claim 1, wherein the coating comprises nano pores.

16. The vaso-occlusive device of claim 15, further comprising a lubricious liquid disposed in the nano pores.

17. A method of manufacturing a vaso-occlusive device according to claim 3, comprising:
cladding a core containing a core material having a first electrical potential with a coating containing a coating material having a second electrical potential to form a wire, wherein the second electrical potential is less than the first electrical potential;
wrapping the wire around a mandrel to form a coil; and
heat-setting the coiled wire.

18. The method of claim 17, wherein the coating material is magnesium.

19. A method of occluding a body cavity with a vaso-occlusive device according to claim 1, comprising:
delivering the vaso-occlusive device to a body cavity; and
inducing a reduction reaction in the body cavity and adjacent the vaso-occlusive device, wherein the reduction reaction reduces infections and promotes cell proliferation in the body cavity.

20. The method of claim 19, further comprising:
delivering a second vaso-occlusive device to the body cavity; and
inducing a second reduction reaction in the body cavity and adjacent the second vaso-occlusive device,
wherein the second vaso-occlusive device does not contain the coating material, and wherein the second reduction reaction reduces infections and promotes cell proliferation in the body cavity.
